(19) United States
(12) Patent Application Publication
(10) Pub. No.: US 2025/0319218 A1
(43) Pub. Date: Oct. 16, 2025

(54) IMPROVED METHOD FOR STERILIZING A PREFILLED SYRINGE (PFS)

(71) Applicant: LABORATOIRE AGUETTANT, LYON (FR)

(72) Inventor: Jeff TONNAR, LYON (FR)

(21) Appl. No.: 18/863,183

(22) PCT Filed: May 15, 2023
(86) PCT No.: PCT/EP2023/062913

§ 371 Date: Nov. 5, 2024

(30) Foreign Application Priority Data
May 16, 2022 (FR) FR2204609

(51) Int. Cl. A61L 2/07; A61M 5/31

(52) U.S. Cl. CPC A61L 2/07; A61M 5/3135; A61L 2202/182; A61L 2202/23; A61L 2202/24

(57) ABSTRACT

A method is provided for applying a sterilization cycle to a prefilled syringe (PFS) according to predetermined parameters (volume of air bubble, value of the back pressure to be applied to the seal after the sterilization tray). The method makes it possible to absolutely guarantee the complete sterilization of the syringe and a return to the initial position of the seal that it had before the application of the sterilization cycle.

### TECHNICAL FIELD

The present invention relates to the field of the packaging of injectable drugs for parenteral administration in liquid form.

More particularly, the invention relates to prefilled syringes (PFS) in which drugs are packaged and which require an aseptic presentation, which is to say that the interface via which the drugs are transferred to the place at which they are to be administered (the site of injection into a patient) needs to be sterile at the moment of use.

The invention seeks to offer a solution for improving the sterilization of an injection device, particularly a PFS syringe, prior to packaging/storing it and in any event prior to using it.

### PRIOR ART

A prefilled syringe is a syringe in which a drug in liquid form has been packaged and sterilized, before or after the filling of the syringe, with a view to storing it, potentially for a long time. This makes it possible to avoid a step of withdrawing it from a bottle or an ampule at the moment of use, so that it can be administered immediately.

This ready-to-use packaging avoids the need to prepare the drug before injecting it and the associated risks of error.

Furthermore, it avoids the risk of microbiological contamination at the time of withdrawal.

In order to prevent the drug from flowing out of the syringe while it is being stored therein, the syringe is plugged at one end of the drug-containing volume by a seal mounted on the plunger of the syringe and, at the other end, by a tip with a removable stopper. The stopper with a frangible obturator may advantageously be like those described respectively in patent applications EP1973592, EP3829681 and that filed on Nov. 26, 2020 under the number FR2012159.

FIG. 1 depicts a prefilled hypodermic syringe 1 according to the prior art, exhibiting symmetry of revolution about a longitudinal axis X.

The syringe 1 comprises a tubular body 2 of cylindrical overall shape with two open longitudinal ends 3, 4.

A rod that forms a plunger 5 passes through the opening of the first end 3 of the tubular body 2. That end of the plunger 5 that is mounted in the tubular body 2 is surmounted by a seal 6. The plunger 5 and the seal 6 form a plunger piston which is designed to slide inside the tubular body 2 along the longitudinal axis X.

The seal 6 that forms the head of the plunger piston is preferably provided with peripheral ribs 60 in direct surface contact, with elastic deformation, with the inside of the body 2 in order to ensure sealing.

The plunger 5 that forms the piston rod enables the user to bring about the translational movement of the plunger piston in order to expel the packaged liquid, once the syringe has been opened.

The tubular body 2 comprises, at the first end 3, a finger flange 7 that projects outwards. Thus, a user presses some digits on the underside of the flange 7, and uses another digit, generally their thumb, to move the plunger 5 and therefore the plunger piston with its seal 6 by pressing on the opposite end 50 of the plunger 5 from the end that forms the plunger piston.

The second end 4 of the tubular body has a wall 8 that is substantially transverse and open at its center and which is extended by a connection tip 9 of the Luer-lock or Luer or NR Fit or EN fit type (or some other specific connection system developed for specific applications eliminating any potential for confusion with the other existing connection systems).

The tip 9 comprises a tubular interior part 10 and an exterior part 11, coaxial with the interior part, which forms a neck and the internal surface of which is threaded for the screwing-on of a tip of complementing shape for connection to a needle or to a drip line or to a catheter or to a device known as a “Luer Activated Device” (LAD) or any other connector available on the market, once the syringe 1 has been opened.

The tubular interior part 10 is of substantially frustoconical shape and opens, at its larger-diameter end which coincides with the opening in the transverse wall 8, onto the interior of the tubular body 2.

A stopper 12 is screwed into the threaded part 11 of the tip 9, thereby obturating the tubular interior part 10 of the tip 9.

The tubular body 2, the plunger piston and its seal 6, the tip 9 and the stopper 12 delimit a sealed space acting as a reservoir for a liquid that is to be administered to a patient. A bubble B of gas, generally air, remains in this sealed space, more specifically in the interior chamber delimited by the body 2.

In order to render the syringe 1 operational, the stopper 12 is unscrewed and this opens the syringe, then a needle or a drip line or a catheter or an LAD or any other commercially-available connector is coupled to the tip 9.

Once the needle or the drip line or the catheter or other commercially-available connector has been introduced into a patient, a user can actuate the plunger 5 thereby administering to the patient the liquid contained in the reservoir.

The main advantage of a prefilled syringe PFS is that of avoiding the user having to transfer a drug from some kind of container into the syringe prior to use. In other words, a prefilled syringe limits the preparation, confusion and risk of contamination at the moment of injection, because all of the contact zones are sterile. Thus, a PFS allows rapid and safe administration of the drug. Such a PES generally has graduations enabling the desired dosage.

Prefilled syringes are often filled and sterilized in a production facility, packaged, then dispatched to a medical facility or a healthcare establishment.

The industrial process for the usual filling and sterilization of a syringe 1 according to FIG. 1 is as follows.

Once the stopper 12 has been fitted and the inside of the body 2 has been filled with the drug in liquid form, the seal 6 is fitted and then the plunger piston 5 is screwed into the seal 6.

The syringe 1 is then packaged in packaging, usually known as a “blister” pack, comprising a thermoformed plastic portion closed by a peelable paper membrane. This paper has the specific feature of being steam-permeable but of constituting a barrier that is substantially impermeable to microorganisms. It may be a specific peelable paper having properties of good permeability with respect to steam and good impermeability with respect to bacteria/microorganisms. Typically, use may be made of a film made of Tyvek®.

Steam sterilization, in order to meet regulatory requirements (to guarantee sterility even in the event of significant initial contamination), has to be performed with at least one sterilization plateau at a temperature of at least 121° C. for at least 15 minutes in what is referred to as “wet” heat, which is to say that all the parts that need to be sterilized need to be in contact with the steam, whether the steam originates from the chamber of an autoclave after having passed through the membrane of the packaging, or from the vaporized content of the syringe. The regulatory requirements also specify that the sterilization cycle can be shorter than 15 minutes if, and only if, the product is unable to withstand 15 minutes at 121° C. In that case, the duration is shorter, provided that the initial bioload can be controlled.

Now, because the annular chambers formed between the lips 60 of the plunger piston 6 are sealed, they are inaccessible to the steam. Sterilization in this region is therefore performed using what is referred to as “dry” heat.

As a result, in order to guarantee sterility in these annular chambers, it is necessary for the sterilization time to be of far longer duration than in the case of “wet” heat (of the order of around 60 minutes rather than 15 minutes). This lengthened sterilization cycle gives rise to three major disadvantages:
markedly higher production cost;
greater degradation of the material of which the syringe body is made, increasing the risk of the release of degradation products (plastic additives or glass components such as aluminum) into the liquid with which the PFS syringe is filled;
impossibility to sterilize at a plateau of 121° C. for 60 minutes for certain active ingredients because they would experience prohibitive degradation.

In order to alleviate these disadvantages, the Applicant Company has proposed a new prefilled syringe PFS as described and claimed in patent EP1919537B1, with steam-passage means, formed and at the rear of the tubular body of the syringe. One of the advantageous embodiments of the passage means consists in at least one groove/notch formed substantially axially in the lateral wall of the syringe body, starting from the interior face and preferably opening outside of the body, locally interrupting an annular bulge formed internally at the proximal end of the chamber internally delimited by the body. During sterilization in an autoclave, the seal of the plunger piston moves back in the tubular body toward the rear thereof until it comes into abutment with a rim in the form of a bulge at the rear of the body intended to prevent it from being ejected. In this position of abutment, the steam can, via the passage means provided, enter the space between the lips of the seal in order to sterilize same. Thereafter, at the end of the sterilization cycle, the seal returns to its initial position, i.e. the position it adopted at the end of filling.

This solution is satisfactory overall because it works reliably for most PFS syringes. Be that as it may, the Applicant Company was able to observe that, for certain types of PFS syringe, sterilization was achieved partially if at all, and/or the seal did not necessarily return to its initial position.

There is therefore still a need to improve still further the method for sterilizing a prefilled syringe PFS, notably in accordance with patent EP1919537B1, particularly in order to absolutely guarantee the desired sterilization and/or the return of the seal to its initial position, once the syringe has been filled.

It is an object of the invention to at least partially address this need.

### SUMMARY OF THE INVENTION

In order to do this, the invention, in one of its aspects, relates to a method for sterilizing a syringe of the prefilled syringe (PFS) type,
a syringe barrel internally comprising an interior chamber filled with a liquid pharmaceutical product to be injected, the interior chamber comprising a proximal end in the vicinity of which there is at least one mechanical blocking means;
a closure cap closing the chamber at its distal end;
a seal arranged in the interior chamber of the barrel forming a head of the plunger piston;

the method comprising a sterilization cycle involving a sterilization plateau, and the following steps:
i) prior to the sterilization cycle, generation within the interior chamber of the barrel of at least a bubble of gas, preferably air or nitrogen, of sufficient volume so as to cause the seal to move back from its initial position that it occupies after filling of the interior chamber with the liquid pharmaceutical product as far as an intermediate sterilization position in which it is in abutment against the mechanical blocking means;

and/or
ii) after the sterilization plateau, the application to the seal of a back-pressure that is great enough to allow the seal to move forward from its intermediate sterilization position more or less as far as its initial position.

According to one advantageous embodiment, the syringe comprises a plunger forming a piston rod to one end and/or of which the seal is fixed, directly or via an adapter, the piston rod being introduced into and able to slide in the body, the fixing of the rod to the adapter or directly to the seal taking place before or after the sterilization cycle.

According to one advantageous embodiment variant, the syringe seal comprises at least two annular sealing lips between which at least one annular chamber is defined, the syringe comprising passage means formed in the body of the syringe and designed to place the annular chamber(s) of the seal in communication with the outside of the body when the seal is in abutment against the mechanical blocking means.

Advantageously, the sterilization plateau is performed at a temperature comprised between 115 and 130° C., preferably at least 121° C. for at least 8 minutes, preferably for at least 15 minutes.

According to one advantageous embodiment of the invention, step i) is performed with an air-bubble volume at least equal to 200 μL.

According to another advantageous embodiment the of invention, step ii) is performed with the seal subjected to a back-pressure at least equal to 1 bar, preferably equal to at least 1.2 bar, more preferably still, equal to at least 1.6 bar.

According to one advantageous embodiment variant, the sterilization cycle comprises, before the sterilization plateau, a phase of continuous heating followed by a phase comprising a plurality of heating pulses achieved by injecting steam and a plurality of evacuation pulses.

As a preference, step ii) being performed during the cooling after the sterilization plateau.

The invention therefore essentially consists in applying a sterilization cycle to a prefilled syringe PFS in accordance with predetermined parameters (air-bubble volume, magnitude of the back-pressure to be applied to the seal after the sterilization plateau) that are able to absolutely guarantee complete sterilization of the syringe and a return of the seal to the initial position that it occupied before the application of said sterilization cycle.

In the context of the invention, these parameters may be adapted according to the type (materials, dimensions) of syringe, seal and potential siliconization thereof.

In an industrial process, the volume of a gas bubble to be created in a PFS syringe will be defined by the following parameters:
dimensions of the syringe;
volume of liquid added to the syringe;
position of the seal with respect to the rear end of the barrel of the syringe.

These parameters are easy to control, allowing standardization of the syringes to be produced according to the invention.

Further advantages and features of the invention will become better apparent from reading the detailed description of exemplary embodiments of the invention given by way of illustrative and nonlimiting example with reference to the following figures.

### BRIEF DESCRIPTION OF THE DRAWINGS

FIG. 1 is a view in longitudinal section of a prefilled syringe according to the prior art in a pre-use filled configuration.

FIG. 2 illustrates, in the form of curves, the movement of the seal during the course of a sterilization cycle for different volumes of air bubble in a PFS syringe in accordance with Patent EP1919537B1.

FIG. 3 illustrates, in the form of curves, the movement of the seal during the course of a sterilization cycle for an air-bubble volume fixed at 200 μL in a PFS syringe in accordance with Patent EP1919537B1.

FIG. 4 illustrates, in the form of curves, the movement of the seal during the course of a sterilization cycle for an air-bubble volume fixed at 1000 μL in a PFS syringe in accordance with Patent EP1919537B1, with cooling after the sterilization plateau.

FIG. 5 illustrates, in the form of curves, the movement of the seal during the course of a sterilization cycle for an air-bubble volume fixed at 1000 μL in a PFS syringe in accordance with Patent EP1919537B1, for different magnitudes of back-pressure applied to the seal, in addition to the intrinsic back-pressure resulting from the cooling after the sterilization plateau.

FIG. 6 illustrates, in the form of curves, the movement of the seal during the course of a sterilization cycle for an air-bubble volume fixed at 800 μL in a PFS syringe in accordance with Patent EP1919537B1, for different magnitudes of back-pressure applied to the seal, in addition to the intrinsic back-pressure resulting from the cooling after the sterilization plateau.

FIG. 7 illustrates, in the form of curves, the movement of the seal during the course of a sterilization cycle for an air-bubble volume fixed at 200 μL in a PFS syringe in accordance with Patent EP1919537B1, for different magnitudes of back-pressure applied to the seal, in addition to the intrinsic back-pressure resulting from the cooling after the sterilization plateau.

### DETAILED DESCRIPTION

The terms “lower”, “upper”, “top”, “bottom” are to be understood with reference to a prefilled syringe configured vertically with the cap above the tubular body of the syringe.

The terms “proximal”, “distal” are to be understood with reference to the grasping of a prefilled syringe. Thus, the cap that closes the syringe is arranged at the distal end thereof.

It must be specified that the scale on the ordinate axis for pressure (bar) and the measured displacement of the seal (mm) is the same.

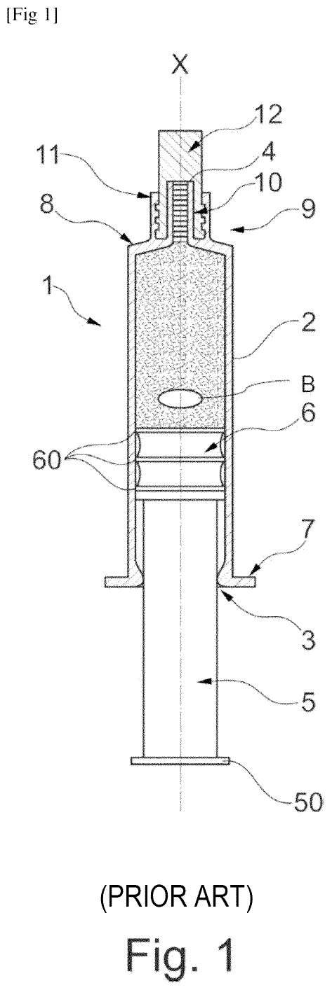

FIG. 1, which relates to a prefilled syringe according to the prior art, has already been described in the preamble. It will therefore not be commented upon below.

The inventors have been able to observe that, for certain types of PFS syringe, such as that in accordance with Patent EP1919537B1, the desired sterilization and/or the return of the seal to its initial position that it occupied once the syringe had been filled, were/was not guaranteed.

The inventors therefore studied the influence of two parameters during sterilization, namely, on the one hand, the size of the air bubble B for causing the seal to move back during the sterilization plateau of the sterilization cycle, and the back-pressure applied to the seal after the sterilization plateau.

The inventors thus carried out a plurality of sterilization tests.

For each test, the syringe with a volume of 10 mL, fitted with its stopper, prefilled and equipped with its rod, was placed in “blister” packaging and then that entity was introduced into an autoclave for the steam sterilization of the syringe.

When the entity was introduced into the autoclave, the seal occupied an initial position defined by the volume of liquid (and the size of the air bubble) prefilled into the barrel body.

It is specified that the seal and the syringe body had been previously siliconized with a medical grade silicone at the time of filling and assembly of the various components.

It is also specified that, for all the tests, the movement of the seal inside the syringe body was measured using a motion sensor marketed under the trade name TMI-Orion NanoVACQ, Serial No. NV130803.

For the tests, the inventor performed additional syringe preparation in order to have perfect knowledge of the volume of the air bubble, by performing the following steps:
filling the interior chamber of the syringe barrel with a volume greater than the nominal volume of the syringe, for example with a volume of 11 mL for a nominal volume equal to 10 mL;
positioning the seal of the syringe in its intended position using the customary tool. An elongate element is introduced from outside the barrel on the seal side so that the excess liquid can escape and leave the syringe by flowing along this elongate element. This then yields a prefilled syringe without an air bubble and with the seal in contact with the liquid;
manually pulling on the plunger piston in order to extract the seal and the rod from the body of the syringe while taking care not to cause the liquid to escape;
withdrawing the desired volume of liquid using a precision pipette. For example, for a bubble volume of 200 μL, 200 μL of liquid is withdrawn;
refitting the seal, using the tool to reposition the seal in the same position as it initially occupied.

These steps culminate in the provision of a PFS syringe filled with a liquid to the nominal volume and containing a calibrated volume of bubble, for example 200 μL.

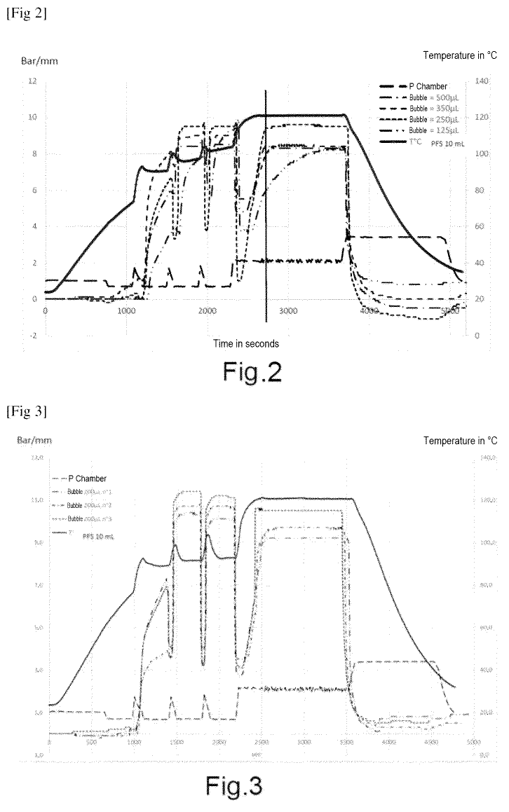

The sterilization cycle applied was as follows:
A/Heating phase lasting approximately 1000 s, so as to raise the temperature in the autoclave.
B/Once the autoclave had reached a temperature of the order of 90° C., application of a plurality of heating pulses, using the injection of steam, and of evacuation pulses. During this phase, the pressure and the temperature simultaneously increase and decrease in the autoclave. That allows the air to be withdrawn from the autoclave and replaced with steam. In general, the more air is withdrawn during this phase, the more effective the sterilization. In this phase B the goal is to cause the seal to move back as far as possible in the barrel body, i.e. until it comes into abutment with the annular bulge. This also allows the air to be withdrawn from the space between the lips of the seal and replaced with steam.
C/Sterilization plateau: once the temperature in the autoclave has reached 121° C., this temperature is maintained at a plateau for around 15 mins (T° C. and pressure both stable). During this phase, the seal needs to be moved back as far as possible in order to allow sterilization of the space between lips of the seal. In FIG. 2, the start of the sterilization plateau is indicated symbolically by the continuous vertical black line.
D/At the end of the sterilization plateau, the autoclave is left to cool. During this cooling, a gas back-pressure either is or is not applied to the seal.

FIG. 2 illustrates the influence of the volume of an air bubble during the sterilization cycle.

It may be seen from this FIG. 2 that:
the seal begins to move back during phase B, i.e. during the application of the vacuum, and moves forward again when steam is injected and there is an increase in pressure;
for bubble volumes of 250, 350 and 500 μL respectively, the backward movement of the seal is at a maximum at the start of the sterilization plateau, and is constant during this plateau,
for a bubble volume of 125 μL, the backward movement of the seal is partial and increases as the sterilization plateau progresses.

From this it may therefore be concluded that somewhere between 125 and 250 μL the bubble volume exhibits a threshold value below which the seal moves backward only partially, and even if maximum backward movement is achieved, this is not until the end of the sterilization plateau. That then means that below this threshold value, sterilization is not completely accomplished, because the space between the lips of the seal is exposed to the sterilizing steam for a shorter length of time.

The inventors therefore ran another series of tests with an air bubble volume fixed at 200 μL. FIG. 3 illustrates the result of this other series of tests. Of the three displacement curves observed, the backward movement of the seal is at a maximum at the start of the sterilization plateau with a discrepancy of 0.6 mm observed in respect of bubble No. 2.

From these tests, the inventors conclude that, by generating an air bubble B of a volume of at least 200 μL for a PFS syringe the interior chamber of which has a volume of 10 mL, maximum backward movement of the seal, i.e. movement until it comes into abutment with the annular bulge is guaranteed and, as a result, complete sterilization is achieved.

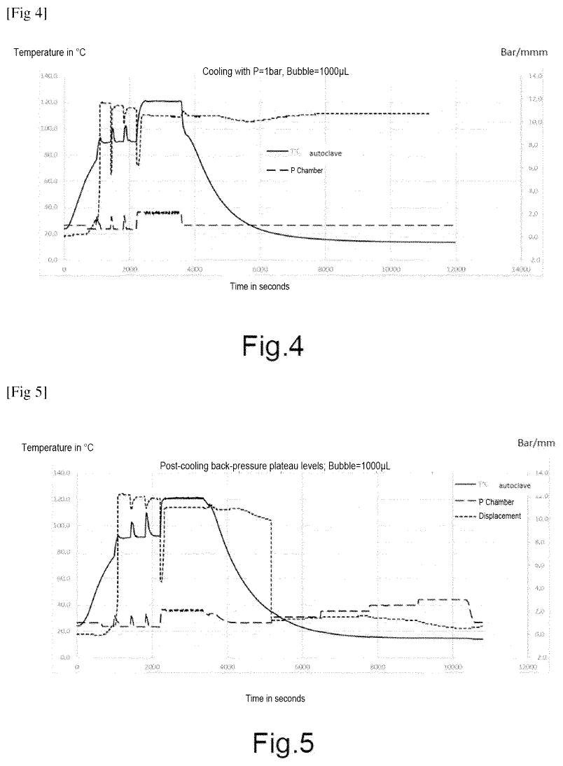

FIG. 4 illustrates a sterilization cycle performed on a 10-ml syringe with a 1000-μL air bubble. For this test, no additional back-pressure was applied to the seal after the sterilization plateau, during cooling. In other words, the back-pressure applied to the seal was that usually associated with the cooling phase. Typically, this back-pressure intrinsic to the cooling diminishes rapidly because it is equal to 1.8 bar at 119° C. and drops to 1.1 bar at 111° C. It is apparent from this FIG. 4 that there is no return of the seal to the initial position it occupied prior to the application of the sterilization cycle, even after an additional 2 h of cooling during which the temperature transitions from 40° C. to 13° C. (and the pressure is equivalent to atmospheric pressure of 1 bar).

FIG. 5 illustrates a sterilization cycle performed on a 10-ml syringe with a 1000-μL air bubble. For this test, no additional back-pressure was applied to the seal after the sterilization plateau, during cooling. Typically, the back-pressure diminishes slowly because it is equal to 1.8 bar at 108° C. and drops to 1.1 bar at 81° C. (drop due to the cooling alone). A back-pressure was then applied once the temperature had dropped below 40° C., with a plateau of 20-mins duration at 1.5 bar, 2.0 bar, 2.5 bar and 3.0 bar, respectively.

It will be noted that when the pressure drops on cooling, it reaches a pressure equal to 1 bar at ambient temperature, and the seal is moved in slightly without reaching a plateau.

By contrast, for the back-pressure plateau at 1.5 bar, the seal is moved in abruptly as far as its initial position.

From this the inventors conclude that with an applied back-pressure at least equal to 1.5 bar and for a 1000-μL air bubble B for a PFS syringe the interior chamber of which has a volume of 10 mL, maximum return of the seal, i.e. return as far as the initial position it adopted before sterilization, possibly to within a few tens of a mm, is guaranteed.

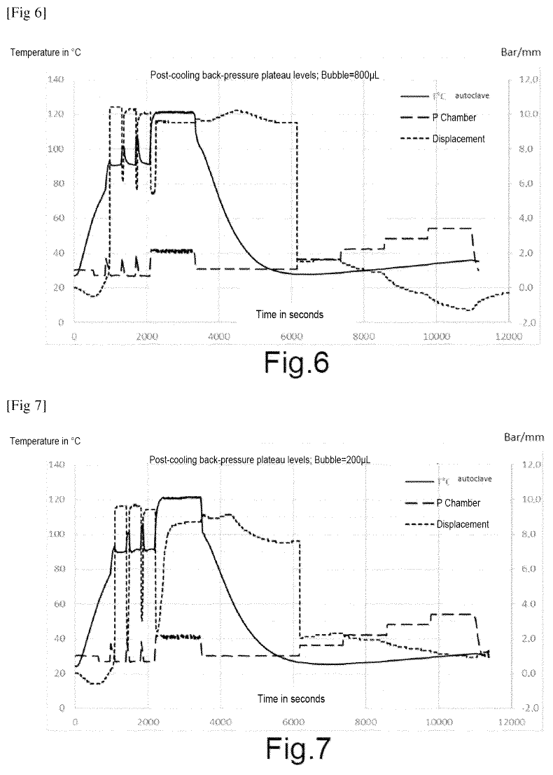

FIG. 6 illustrates a sterilization cycle performed on a 10-mL syringe with an 800-μL air bubble. For this test, no additional back-pressure was applied to the seal after the sterilization plateau, during cooling. Typically, this applied back-pressure diminishes rapidly because it is equal to 1 bar at 115° C. An increased back-pressure was additionally applied once the temperature had dropped below 40° C., with a plateau of 20-mins duration at 1, 1.6 bar, 2.2 bar, 2.8 bar, and 3.4 bar, respectively.

It may be noted that with a pressure equal to 1 bar (atmospheric pressure), the seal remains moved back.

By contrast, for the back-pressure plateau at 1.6 bar the seal is moved in abruptly almost as far as the initial position it occupied, reaching a plateau. There is also an additional inward movement, without reaching a plateau, with a return of the seal to its initial position during the course of a 2.2 bar plateau. The inward movement exceeds the initial position after 5 minutes at 2.2 bar.

From this the inventors conclude that with an applied back-pressure at least equal to 1.6 bar and for an 800-μL air bubble B for a PFS syringe the interior chamber of which has a volume of 10 mL, maximum return of the seal, i.e. return as far as the initial position it adopted before sterilization, possibly to within a few tens of a mm, is guaranteed.

FIG. 7 illustrates a sterilization cycle performed on a 10-ml syringe with a 200-μL air bubble. For this test, no additional back-pressure was applied to the seal after the sterilization plateau, during cooling. Typically, this pressure diminishes rapidly because it is equal to 1 bar at 117° C. An increased back-pressure is then applied once the temperature has dropped below 40° C., with a plateau of 20-mins duration at 1, 1.6 bar, 2.2 bar, 2.8 bar, and 3.4 bar, respectively.

It may be noted that with an applied back-pressure equal to 1 bar, the seal is pushed in with a plateau at 7.5 mm.

By contrast, for the back-pressure plateau at 1.6 bar the seal is moved in abruptly almost as far as its initial position, reaching a plateau. There is also an additional inward movement, without reaching a plateau, with a return of the seal to its initial position during the course of a 2.2 bar plateau. The inward movement exceeds the initial position after 5 minutes at 2.2 bar.

From this the inventors conclude that with an applied back-pressure at least equal to 1.6 bar and for a 200-μL air bubble B for a PFS syringe the interior chamber of which has a volume of 10 mL, maximum return of the seal, i.e. return as far as the initial position it adopted before sterilization, possibly to within a few tens of a mm, is guaranteed.

Therefore the application of a suitable back-pressure to the seal, in addition to the back-pressure intrinsic to the cooling phase, returns the seal to its initial position.

Other variants and embodiments may be envisaged without departing from the scope of the invention.

Thus, the sufficient volume of the gas bubble and/or the level of back-pressure to be applied to the seal will need to be determined notably on the basis of the interior coatings of the barrel of the syringe, of the seal and on the basis of the shape and material of the latter and the possible siliconization of the surfaces.

1. A sterilization method for sterilizing a syringe of the prefilled syringe (PFS) type, having
a syringe barrel internally comprising an interior chamber filled with a liquid pharmaceutical product to be injected, the interior chamber comprising a proximal end in the vicinity of which there is at least one mechanical blocking means
a closure cap closing the chamber at its distal end;
a seal arranged in the interior chamber of the barrel forming a head of the plunger piston;
the method comprising:
a sterilization cycle involving a sterilization plateau, and the following steps of:
i) prior to the sterilization cycle, generation within the interior chamber of the barrel of at least a bubble of gas, of sufficient volume so as to cause the seal to move back from its initial position that it occupies after filling of the interior chamber with the liquid pharmaceutical product as far as an intermediate sterilization position in which it is in abutment against the mechanical blocking means; and/or
ii) after the sterilization plateau, the application to the seal of a back-pressure that is great enough to allow the seal to move forward from its intermediate sterilization position more or less as far as its initial position.

2. The sterilization method as claimed in claim 1, the syringe comprising a plunger forming a piston rod to one end of which the seal is fixed, directly or via an adapter, the piston rod being introduced into and able to slide in the body, the fixing of the rod to the adapter or directly to the seal taking place before or after the sterilization cycle.

3. The sterilization method as claimed in claim 1, the syringe seal comprising at least two annular sealing lips between which at least one annular chamber is defined, the syringe comprising passage means formed in the body of the syringe and designed to place the annular chamber(s) of the seal in communication with the outside of the body when the seal is in abutment against the mechanical blocking means.

4. The sterilization method as claimed in claim 1, the mechanical blocking means being an annular bulge formed internally inside the barrel.

5. The sterilization method as claimed in claim 1, the sterilization plateau being performed at a temperature comprised between 115 to 130° C., for at least 8 minutes.

6. The sterilization method as claimed in claim 1, step i) being performed with an air-bubble volume at least equal to 200 μL.

7. The sterilization method as claimed in claim 1, step ii) being performed with the seal subjected to a back-pressure at least equal to 1 bar, preferably equal to at least 1.2 bar, more preferably still, equal to at least 1.6 bar.

8. The sterilization method as claimed in claim 1, the sterilization cycle comprising, before the sterilization plateau, a phase of continuous heating followed by a phase comprising a plurality of heating pulses achieved by injecting steam and a plurality of evacuation pulses.

9. The sterilization method as claimed in claim 1, step ii) being performed during the cooling after the sterilization plateau.

10. The sterilization method as claimed in claim 1, wherein said at least a bubble of gas is air or nitrogen.

11. The sterilization method as claimed in claim 5, the sterilization plateau being performed at a temperature of least 121° C.

12. The sterilization method as claimed in claim 1, the sterilization plateau being performed for at least 15 minutes.

13. The sterilization method as claimed in claim 7, wherein the back-pressure is at least equal to at least 1.2 bar.

14. The sterilization method as claimed in claim 7, wherein the back-pressure is at least equal to at least 1.6 bar.
